(19) United States
(12) Patent Application Publication
(10) Pub. No.: US 2025/0339350 A1
(43) Pub. Date: Nov. 6, 2025

(54) Dental Compositions and Methods of Making and Using Same

(71) Applicant: Solventum Intellectual Properties Company, Maplewood, MN (US)

(72) Inventors: Yizhong Wang, Woodbury, MN (US); Afshin Falsafi, Woodbury, MN (US); Joel D. Oxman, Minneapolis, MN (US)

(21) Appl. No.: 18/862,273

(22) PCT Filed: May 22, 2023
(86) PCT No.: PCT/IB2023/055248

§ 371 Date: Nov. 1, 2024

Related U.S. Application Data
(60) Provisional application No. 63/365,356, filed on May 26, 2022.

(51) Int. Cl. A61K 6/891; A61K 6/79; A61K 6/836

(52) U.S. Cl. CPC A61K 6/891; A61K 6/79; A61K 6/836

(57) ABSTRACT

A multi-part hardenable dental composition. A first part includes a mono- and/or multi-functional component having one or more ethylenically unsaturated group(s), acid-reactive glass particles, and an oxidizing agent. A second part includes a mono- and/or multi-functional component having one or more ethylenically unsaturated group(s), a water miscible polyacid, a reducing agent that includes an aromatic tertiary amine, an acid having a pKa less than that of carboxylic acid, and non-reactive filler.

### BACKGROUND

Two-part dental cements have been described in, for example, U.S. Pat. No. 6,982,288, International Pub. WO 2020/075007, and International Pub. WO 2011/081975.

### DETAILED DESCRIPTION

Two-part glass ionomer cements have been in dental use for some time. Such materials are comprised of an ionic polymer component and a reactive glass component, which when mixed together in the presence of water undergo a cement setting reaction. These dental materials provide several desirable attributes including prolonged fluoride release, tolerance to moisture and saliva, good mechanical properties and excellent adhesion to dental hard tissues without pretreatments such as conditioners or adhesives. Powder-liquid, powder-paste, paste-paste, paste-liquid, and liquid-liquid two-part cements have been reported.

Various drawbacks have become evident with known materials and methods, including, for example, mechanical strength variability, varying consistencies, unsatisfactory working or setting times, cost per application, multiple dispensing and mixing steps, mechanical mixing equipment, and shelf life.

The materials described above are available as multi-part systems, typically in two-parts. These can be in any combination of powder, liquid, or paste. Shelf stability of the individual parts is extremely important so that there is no change in viscosity, color, or any other property occurring during the shelf life of the material (typically 2-4 years). In use, the parts are mixed together and then applied. Setting should occur in a short period of time so that the procedure is not uncomfortable for the patient or the operator. The setting characteristics should allow for sufficient time for mixing the materials and applying to the tooth preparation and/or prosthodontic or orthodontic device in place in the mouth.

In prior multi-part glass ionomer cement systems, a reducting agent (such as an aromatic tertiary amine) is paired in one of the parts with an oxidizing agent (such as a peroxide). In such systems, with the electron rich structure on the reducing agent, undesirable self-cure of resin with reactive monomer and polymers over relatively short storage periods may occur. Consequently, there continues to be a growing interest in alternative methods and compositions for delivering glass ionomer cements and related materials in a more stable manner.

### Definitions

The term “water soluble” refers to a material, such as a monomer, which is partially or fully water soluble and dissolves in water alone in the amount of at least 5 g per liter of water at 25° C..

The term “comprising” and variations thereof (e.g., comprises, includes, etc.) do not have a limiting meaning where these terms appear in the description and claims.

As used herein, “a,” “an,” “the,” “at least one,” and “one or more” are used interchangeably, unless the context clearly dictates otherwise.

Also herein, the recitations of numerical ranges by endpoints include all numbers subsumed within that range (e.g., the range of viscosity ratios 1:0.06 to 1:13 includes 1:0.06 to 1:13, 1:0.1 to 1:13, 1:0.25 to 1:13, 1:0.5 to 1:13, 1:0.6 to 1:13, 1:1 to 1:13, 1:0.06 to 1:10, 1:0.06 to 1:7.5, 1:0.06 to 1:5, 1:0.06 to 1:3.5, 1:0.06 to 1:1, 1:0.1 to 1:10, 1:0.25 to 1:7.5, 1:0.5 to 1:5, 1:0.6 to 1:3.5, 1:0.75 to 1:2, 1:0.9 to 1:1.1, etc.).

In some embodiments, the present disclosure is directed to a multi-part (e.g., two-part) hardenable dental composition (e.g., glass ionomer cement), the first part including (i) liquid (at room temperature) monofunctional and/or multifunctional monomers, oligomers, or polymers, (ii) an oxidizing agent; and (iii) acid-reactive glass, and the first part being free or substantially free of water; and the second part including (i) liquid monofunctional and/or multifunctional monomers, oligomers, or polymers, (ii) a reducing agent; (iii) a strong acid; (iv) a polyacid with or without a polymerizable sidechain group, and (v) water.

Surprisingly, it was discovered that by separating the components of the ionic redox polymerization system (i.e., the reducing agent and the oxidizing agent) into different parts, a more reliable two-part cement system could be achieved. That is, it was discovered that by pairing the reducing agent and polyacid in a second part (and pairing the reducing agent in the second part with another strong acid to form a latent catalyst), and by not including water (or at least not an appreciable amount of water) in the first part (that includes the oxidizing agent) a composition having all the benefits of known two-part cement compositions, but with enhanced stability (e.g., shelf life stability, even when subjected to handling errors (e.g., potential water loss due to loose fitting caps or failure to recouple cap to container in a timely fashion)) may be obtained.

In some embodiments, a first part of the multi-part hardenable composition may include (i) liquid (at room temperature) mono- and/or multi-functional components having ethylenically unsaturated group(s) (sometimes referred to herein as a “resin system”), (ii) an oxidizing agent; and (iii) acid-reactive glass. In some embodiments, the first part may include no more than a small amount of water.

In some embodiments, the first part may be in the form of a paste. That is, not a powder or a liquid, but a mixture of liquid and non-dissolvable powder/solid components having a generally uniform composition.

In some embodiments, the first part may include either or both of liquid mono- and multi-functional (e.g., di-functional) components having ethylenically unsaturated group(s). The liquid mono- or multi-functional components may include monomers, oligomers, or polymers. In some embodiments, the liquid mono- or multi-functional components may be water soluble. The liquid mono- or multi-functional components may also be selected such that they are miscible with the other components of the hardenable composition. That is, these components may be at least sufficiently miscible that they do not undergo substantial sedimentation when combined with the other parts of the composition. In some embodiments, the first part may include both liquid mono- and multi-functional (e.g., di-functional) components having ethylenically unsaturated group(s).

In some embodiments, suitable ethylenically unsaturated groups include allyl, vinyl, acrylate, and methacrylate groups. In some embodiments, such monomers (or oligomers or polymers) have a relatively low molecular weight and include only one ethylenically unsaturated group per monomer molecule. In some embodiments, the molecular weight of such monomers is about 100 to about 1000. In some embodiments, including any one of the above embodiments which includes a water-soluble liquid mono-functional monomer having one ethylenically unsaturated group per monomer molecule, the water soluble liquid monomer may be selected from the group consisting of 2-hydroxyethyl (meth)acrylate, glycerol mono(meth)acrylate, sugar methacrylates, and a combination thereof.

Also as indicated above, the multi-part hardenable compositions described herein, in certain embodiments may also include a component having at least two ethylenically unsaturated groups per monomer molecule which provides some cross linking in the composition when hardened. In some embodiments, this monomer may have a viscosity less than bisphenol A-glycidyl methacrylate (Bis-GMA), or not more than 50 percent of Bis-GMA.

In some embodiments, suitable difunctional monomers may include glycerol dimethacrylate. Alternatively or additionally, a water soluble monomer is used. Suitable water soluble (dimeth)acrylates include various molecular weights of polyethylene glycol (dimeth)acrylates ranging from approximately 400 to 1000 weight average molecular weight.

The components of the resin system may be selected such that they are miscible with the other components of the hardenable composition. That is, the components of the resin system may be at least sufficiently miscible that they do not undergo substantial sedimentation when combined with the other ingredients of the composition (e.g., reducing agent and oxidizing agent). In some embodiments, the components of the resin system are miscible with water. The components of the resin system can be monomers, oligomers, polymers, or combinations thereof.

In some embodiments, liquid mono- and multi-functional (e.g., di-functional) components having ethylenically unsaturated group(s) may be present in the first part in an amount of between 1 and 50 wt. %, between 3 and 40 wt. %, or between 5 and 30 wt. %, based on the total weight the first part.

In some embodiments, suitable oxidizing agents may include persulfates such as sodium, potassium, ammonium and alkyl ammonium persulfates, benzoyl peroxide, hydroperoxides such as cumene hydroperoxide, tert-butyl hydroperoxide, tert-amyl hydroperoxide and 2,5-dihydroperoxy-2,5-dimethylhexane, salts of copper (II) and iron (III), hydroxylamine, perboric acid and its salts, salts of a permanganate anion, and combinations thereof. Hydrogen peroxide can also be used, although it may, in some instances, interfere with the photoinitiator, if one is present. In some embodiments, the oxidizing agent may include potassium persulfate (e.g., milled potassium persulfate). The oxidizing agent may optionally be provided in an encapsulated form as described in U.S. Pat. No. 5,154,762. The oxidizing agent may be selected such that it is miscible in the compositions and/or miscible in water.

In some embodiments, the oxidizing agent may be present in the first part in an amount of between 0.01 and 10 wt. %, between 0.1 and 4.0 wt. %, or between 0.5 and 2.0 wt. %, based on the total weight the first part.

In some embodiments, the first part may include acid-reactive glass. Suitable acid-reactive glasses may include ion-leachable glasses, e.g., as described in U.S. Pat. Nos. 3,655,605; 3,814,717; 4,143,018; 4,209,434; 4,360,605 and 4,376,835. In some embodiments, the acid-reactive glass may be selected from borate glasses, phosphate glasses and fluoroaluminosilicate glasses. In some embodiments, the acid-reactive glass may include fluoroaluminosilicate (FAS) glass.

In embodiments that include FAS glass, the FAS glass may include sufficient elutable cations so that a hardened dental composition will form when the glass is mixed with the components of the hardenable composition. The glass may also include sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass may be in the form of particles that are sufficiently finely divided so that they can conveniently be mixed with the other cement components and will perform well when the resulting mixture is used in the mouth.

In some embodiments, the average particle size (average longest dimension—typically, diameter) for the FAS glass is no greater than 10 micrometers or no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT and KETAC-FIL (3M ESPE Dental Products, St. Paul, MN), FUJI II, GC FUJI LC and FUJI IX (G-C Dental Industrial Corp., Tokyo, Japan) and CHEMFIL Superior (Dentsply International, York, PA). Mixtures of fillers can be used if desired.

In some embodiments, the FAS glass can be subjected to a surface treatment. Suitable surface treatments include acid washing (e.g., treatment with a phosphoric acid), treatment with a phosphate, treatment with a chelating agent such as tartaric acid, and treatment with a silane or an acidic or basic silanol solution. Desirably the pH of the treating solution or the treated glass is adjusted to neutral or near-neutral, as this can increase storage stability of the hardenable composition.

In some embodiments, any of the above described acid-reactive glass particles may also be subjected to a surface treatment. Suitable surface treatments include acid washing, treatment with phosphates, treatment with chelating agents such as tartaric acid, treatment with a silane or silanol coupling agent. In some embodiments, the acid-reactive glass particles may be silanol treated fluoroaluminosilicate glass particles, as described in U.S. Pat. No. 5,332,429, the disclosure of which is incorporated by reference herein in its entirety.

In some embodiments, the acid-reactive glass may be present in the first part in an amount of between 1 and 80 wt. %, between 3 and 60 wt. %, or between 5 and 40 wt. %, or based on the total weight the first part.

In some embodiments, the first part may not include water or be substantially devoid of water. In this regard, in some embodiments, the first part may include water in an amount of less than 5 wt. %, less than 1 wt. %, or less than 0.5 wt. %, based on the total weight of the first part.

In some embodiments, a second part of a multi-part hardenable composition may include (i) liquid monofunctional and/or multifunctional monomers, oligomers, or polymers, (ii) a polyacid with or without a polymerizable sidechain group; (iii) a reducing agent; (iv) a strong acid, and (v) water. As with the first part, in some embodiments, the second part may be in the form of a paste.

In some embodiments, the second part may include either or both of liquid mono- and multi-functional (e.g., di-functional) components having ethylenically unsaturated group(s). The liquid mono- or multi-functional components may include monomers, oligomers, or polymers. Generally (but independently), the liquid mono- and multi-functional (e.g., di-functional) components of the second part may be of the same types discussed with respect to the first part.

Additionally, in some embodiments, the resin system of the second part may include one or more acid-functional monomers, oligomers, or polymers (also referred to herein as acid functional components). Such components may be ethylenically unsaturated compounds with acid functionality and may include oxyacid functional derivatives of carbon, phosphorous, sulfur, and boron compounds, and may be selected from those described in U.S. Pat. No. 7,156,911, Columns 6-7, the entire disclosure of which is incorporated by reference herein in its entirety. In some embodiments, the acid-functional components may include polymers, including homopolymers and copolymers (i.e., of two or more different monomers), of alkenoic acids such as acrylic acid, 2-chloroacrylic acid, 2-cyanoacrylic acid, aconitic acid, citraconic acid, fumaric acid, glutaconic acid, itaconic acid, maleic acid, mesaconic acid, methacrylic acid, and tiglic acid.

In some embodiments, liquid mono- and multi-functional (e.g., di-functional) components having ethylenically unsaturated group(s) may be present in the second part in an amount of between 1 and 50 wt. %, between 3 and 40 wt. %, or between 5 and 30 wt. %, based on the total weight the second part.

In some embodiments, the second part may include a polyacid. In some embodiments, the polyacid may be water miscibile. Suitable water miscible polyacids may include homo- or copolymers of unsaturated mono-, di-, and tricarboxylic acids, for example, homo- or copolymers of acrylic acid, itaconic acid and maleic acid. In some embodiments, the water miscible polyacids may include a polymer having sufficient pendent ionic groups to undergo a setting reaction in the presence of a reactive glass and water, and sufficient pendent non-ionically polymerizable groups to enable the resulting mixture to be cured by a redox curing mechanism and/or by exposure to radiant energy.

In some embodiments, the polyacid may be a functional acidic (co)polymer (the reaction product of a polyacid with a coupling agent). In some embodiments, the polyacid may be the reaction product of a polymer selected from the group consisting of polyacrylic acids, copolymers of acrylic and itaconic acids, copolymers of acrylic and maleic acids, copolymers of methyl vinyl ether and maleic anhydride or maleic acid, copolymers of ethylene and maleic anhydride or maleic acid, copolymers of styrene and maleic anhydride or maleic acid, and a combination thereof, with a coupling compound selected from the group consisting of acryloyl chloride, methacryloyl chloride, vinyl azalactone, allylisocyanate, 2-hydroxyethyl methacrylate, 2-aminoethyl methacrylate, and 2-isocyanatoethyl methacrylate. In some embodiments, the polyacid may be the reaction product of 2-isocyanatoethyl methacrylate and a copolymer of acrylic acid and itaconic acid prepared as described in U.S. Pat. No. 5,130,347, Example 11 (an acrylic-itaconic acid copolymer with pendant methacrylate side group).

In some embodiments, the polyacid may have Formula I:

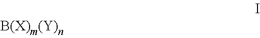

wherein B is an organic backbone, each X independently is an ionic group which can undergo a setting reaction in the presence of water and the acid-reactive glass particles, each Y independently is a non-ionically polymerizable group, m is at least 2, and n is at least 1. In some embodiments, X is —COOH and Y is an ethylenically unsaturated group. In some embodiments, the backbone B is an oligomeric or polymeric backbone of carbon-carbon bonds, optionally containing non-interfering substituents such as oxygen, nitrogen or sulfur heteroatoms. The term “non-interfering” refers to substituents or linking groups that do not unduly interfere with either the ionic or the non-ionic polymerization reaction. In some embodiments, B is a hydrocarbon backbone. X and Y groups can be linked to the backbone B directly or by means of any non-interfering linking group, such as substituted or unsubstituted alkylene, alkyleneoxyalkylene, arylene, aryleneoxyalkylene, alkyleneoxyarylene, arylenealkylene, or alkylenearylene groups. Alkylene and arylene refer to the divalent forms of alkyl and aryl, respectively. The linking group may also include linkages such as —OC(═O)—, —C(═O)NH—, —NH—C(═O)O—, —O—, and the like, and combinations thereof, wherein each of these may be used in either direction. In some embodiments, Y is attached to B via an amide linkage. In some embodiments, Y is an acryloyloxy, methacryloyloxy, acrylamido, or methacrylamido group.

The polyacid of Formula I can be prepared according to a variety of synthetic routes, including, but not limited to, (1) reacting n X groups of a polymer of the formula B(X)ₘ₊ₙ with a suitable compound in order to form n pendent Y groups, (2) reacting a polymer of the formula B(X)ₘ at positions other than the X groups with a suitable compound in order to form n pendent Y groups, (3) reacting a polymer of the formula B(Y)ₘ₊ₙ or B(Y)ₙ, either through Y groups or at other positions, with a suitable compound in order to form m pendent X groups and (4) copolymerizing appropriate monomers, e.g., a monomer containing one or more pendent X groups and a monomer containing one or more pendent Y groups. The synthetic route (1) above is preferred. Such groups can be reacted by the use of a “coupling compound”, i.e., a compound containing both a Y group and a reactive group capable of reacting with the polymer through an X group, thereby covalently linking the Y group to the backbone B in a pendent fashion. Suitable coupling compounds are organic compounds, optionally containing non-interfering substituents and/or non-interfering linking groups between the Y group and the reactive group.

As indicated above, coupling compounds suitable for preparing polyacids for use herein include compounds that contain at least one group capable of reacting with X in order to form a covalent bond, as well as at least one polymerizable ethylenically unsaturated group. When X is carboxyl, a number of groups are capable of reacting with X, including both electrophilic and nucleophilic groups. Examples of such groups include hydroxyl, amino, isocyanato, halo carboxyl, and oxiranyl. Examples of suitable coupling compounds include, but are not limited to, acryloyl chloride, methacryloyl chloride, vinyl azalactone, allylisocyanate, 2-hydroxyethyl methacrylate, 2-aminoethyl methacrylate, and 2-isocyanatoethyl methacrylate. Other examples of suitable coupling compounds include those described in U.S. Pat. Nos. 4,035,321 and 5,814,682, the disclosures of which are hereby incorporated by reference.

In some embodiments, polyacid may be present in the second part in an amount of between 2 and 50 wt. %, between 5 and 35 wt. %, or between 10 and 15 wt. %, based on the total weight the second part.

In some embodiments, suitable reducing agents may include ascorbic acid, metal complexed ascorbic acid, aromatic amines such as dimethylaminophenethanol and dihydroxyethyl-p-toludine, cobalt (II) chloride, ferrous chloride, ferrous sulfate, hydrazine, hydroxylamine, oxalic acid, thiourea, alkyl thioureas and salts of a dithionite, 1-allyl-2-thiourea, thiosulfate, aromatic sulfinic acid salts such as benzene sulfinic salts and p-toluenesulfinic salts, sulfite anion and a combination thereof.

In some embodiments, the reducing agent may include a tertiary aromatic amine. In some embodiments, the tertiary aromatic amine may have a structural formula as follows:

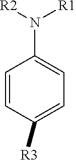

where R1, R2, R3 are, independently, a hydrogen atom, or an alkyl group, alkyl alcohol group, an alkyl group that includes an ester or amide linkage, an ester group, an amide group, or a utethane group, having from 1-8, 1-6, or 1-4 carbon atoms. In some embodiments, the tertiary aromatic amine may be selected from:

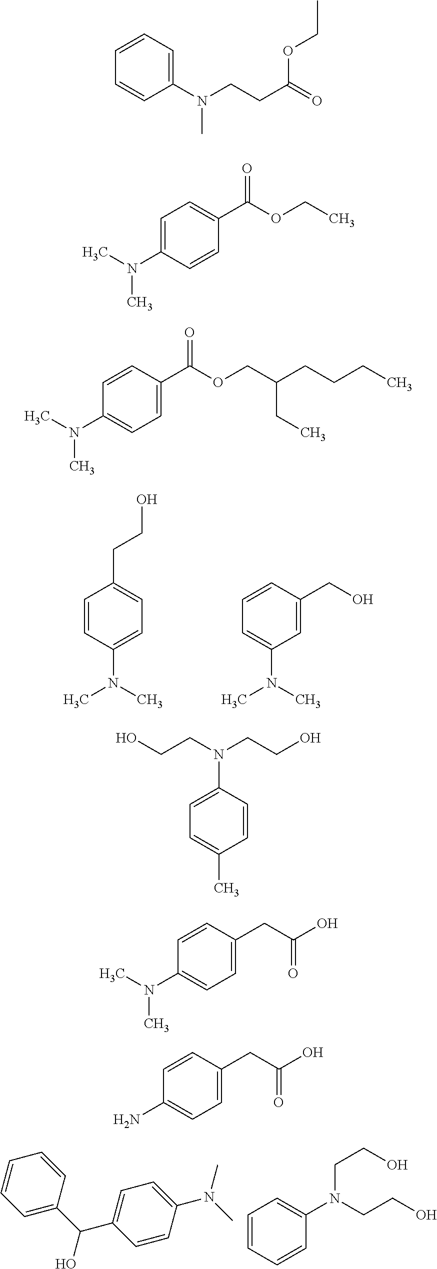

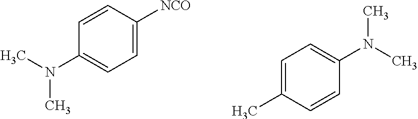

or any derivative of such tertiary aromatic amines.

In some embodiments, the reducing agent (e.g., tertiary aromatic amine) may be present in the second part in an amount of between 0.1 and 8.0 wt. %, between 0.25 and 5.0 wt. %, or between 0.5, and 3.0 wt. %, based on the total weight the second part.

As previously discussed, the second part may include a strong acid in addition to the reducing agent to form a latent catalyst for the multi-part hardenable composition. Generally, the strong acid may operate suppress the reactivity of the reducing agent. More specifically, the strong acid may react with the reducing agent (e.g., an aromatic tertiary amine) in water to form a salt (e.g., an amine salt), dramatically reducing the electron density on the nitrogen atom with the bonding to a proton, thereby reducing the reducing capability of the aromatic tertiary amine to a form free radical. This, in turn, results in the avoidance of undesirable/premature self curing of the second part. In some embodiments, suitable strong acids may be those that are stronger than carboxylic acid (i.e., acids having a pKa less than that of carboxlic acid). In some embodiments, suitable strong acids may include phosphoric acid, sulfate acid, nitric acid, hydrochloride acid, and monomers containing one or more phosphate groups, such as methacryloyloxydecyl dihydrogen phosphate (MDP) or methacryloyloxyhecyl dihydrogen phosphate (MHP).

It is to be appreciated that, in some embodiments, after mixing of the first part and the second part, the reducing agent, which had reacted with the strong acid to form a salt, may be regenerated. That is, after mixing, the aqueous based resin system and polyacid of the second part will be paired with the resin system and the oxidizing agent of the first part, the oxidizing agent may be dissolved in the water of the second part, and (in embodiments employing an tertiary aromatic amine as reducing agent) the aromatic tertiary amine phosphate salt (of the second part) can convert to the aromatic tertiary amine after mixing with the acid-reactive filler and any other in the first part. The regenerated aromatic tertiary amine may then dissolve in the water/resin solution to form the normal peroxide/amine redox pair and form free radicals to initiate curing of the multi-part hardenable composition.

In some embodiments, the second part may include water. Water may be present in the second part in an amount of between 1 and 40 wt. %, between 2 and 30 wt. %, or between 5 and 20 wt. %, based on the total weight the second part.

In some embodiments, non-reactive fillers may also be included in the compositions described herein to control viscosity as well as for other reasons, such as to achieve a desired appearance, impart desired strength properties, impart radiopacity, and the like. In some embodiments, including any one of the above embodiments, the first part, the second part, or both, may further include a non-reactive filler in an amount of between 5 and 60 wt. %, between 10 and 50 wt. %, or between 15 and 40 wt. %, based on the total weight the respective part.

In some embodiments, non-reactive fillers may be selected from one or more materials suitable for incorporation in compositions used for medical applications, such as fillers currently used in dental restorative compositions and the like. In some embodiments, the filler may have a maximum particle diameter less than 50 micrometers and an average particle diameter less than about 10 micrometers. The filler can have a unimodal or polymodal (e.g., bimodal) particle size distribution.

In some embodiments, the non-reactive filler is selected from the group consisting of inorganic material, crosslinked organic material, and a combination thereof. Suitable crosslinked organic materials are insoluble in the composition, and are optionally filled with inorganic filler. The filler should be non-toxic and suitable for use in the mouth. The filler can be radiopaque, radiolucent or non-radiopaque.

Examples of suitable non-reactive inorganic fillers are naturally-occurring or synthetic materials such as quartz, nitrides (e.g., silicon nitride), glasses derived from, for example, Ce, Sb, Sn, Zr, Sr, Ba and Al, colloidal silica, colloidal zirconia, feldspar, borosilicate glass, kaolin, talc, titania, and zinc glass; low Mohs hardness fillers such as those described in U.S. Pat. No. 4,695,251; and submicron silica particles (e.g., pyrogenic silicas such as the “Aerosil” Series “OX 50”, “130”, “150” and “200” silicas sold by Degussa and “Cab-O-Sil M5” silica sold by Cabot Corp.); metallic powders such as those disclosed in U.S. Pat. No. 5,084,491, especially those disclosed at column 2, lines 52-65; and combinations thereof.

Examples of suitable non-reactive organic filler particles include filled or unfilled pulverized polycarbonates, polyepoxides, and the like. Preferred non-reactive filler particles are quartz, submicron silica and zirconia, and non-vitreous microparticles of the type described in U.S. Pat. No. 4,503,169. Mixtures of these non-reactive fillers are also contemplated, as well as combination fillers made from organic and inorganic materials.

In some embodiments which include a non-reactive filler, the non-reactive filler is selected from the group consisting of fumed silica, zirconia-silica, quartz, nonpyrogenic silica, and combinations thereof.

In some embodiments, the surface of the non-reactive filler particles may be treated with a coupling agent in order to enhance the bond between the filler and polymerizable components when the composition is hardened. The use of suitable coupling agents include gamma-methacryloxypropyltrimethoysilane, gamma-mercaptopropyltriethoxysilane, gamma-aminopropyltrimethoxysilane, SILQUEST A-1230 (Momentive Performance Chemicals), and the like.

Additional components, which are suitable for use in the oral environment, may optionally be used in (either or both parts of) the multi-part hardenable compositions described herein. In one example, such components include solvents, cosolvents (e.g., alcohols) or diluents. In another example, indicators, dyes, pigments, inhibitors, accelerators, viscosity modifiers, wetting agents, tartaric acid, chelating agents, surfactants, buffering agents, stabilizers (including free-radical stabilizers), submicron silica particles, additives that impart fluorescence and/or opalescence, modifying agents that prolonged working time, and other materials that will be apparent to those skilled in the art may be used. Additionally, medicaments or other therapeutic substances can be optionally added to the compositions. Examples include whitening agents, breath fresheners, flavorants, fragrances, anticaries agents (e.g., xylitol), fluoride sources, remineralizing agents (e.g., calcium phosphate compounds), enzymes, anesthetics, clotting agents, acid neutralizers, chemotherapeutic agents, immune response modifiers, thixotropes, polyols, anti-inflammatory agents, antimicrobial agents, antifungal agents, agents for treating xerostomia, desensitizers, and the like of the type which may be used in dental compositions. Combinations of any of the above additives may also be used in the compositions described herein. The selection and amount of any one such additive can be determined by one of skill in the art according to the desired result.

Modifying agents which may prolong the time between the beginning of the setting reaction in a restoration and the time sufficient hardening has occurred to allow subsequent clinical procedures to be performed on the surface of the restoration include, e.g., alkanolamines such as ethanolamine and triethanolamine, and mono-, di-, and tri-sodium hydrogenphosphates. Modifying agents can be added to either part A or part B. When used, they are present at a concentration between about 0.1 to 10 percent by weight, based on the total composition weight.

Certain stabilizers provide color stability. Such stabilizers include oxalic acid, sodium metabisulfite, sodium bisulfite, sodium thoisulfate, metaphosphoric acid, and combinations thereof.

Free radical stabilizers can be used with a photoinitiator to prevent premature polymerization or to adjust the working time in free radically initiated compositions. Suitable examples of free radical stabilizers include, e.g., butylated hydroxytoluene (BHT) and methyl ethyl hydroquinone (MEHQ).

Viscosity modifiers include thickening agents. Suitable thickening agents include hydroxypropyl cellulose, hydroxymethyl cellulose, carboxymethylcellulose and its various salts such as sodium, and combinations thereof.

In some embodiments, either or both to the first and second parts may include a photoinitiator. In some embodiments, photoemitter may be present not as a primary curing enabler but, rather, to allow a practitioner to expeditiously cure excess portions of the hardenable composition during use (e.g., an excess amount of the material that is present once a dental crown has been seated on a prepared tooth).

Generally, the photoinitiators may act as a source of free radicals when activated by heat or light. Such initiators can be used alone or in combination with one or more accelerators and/or sensitizers. Suitable photoinitiators (i.e., photoinitiator systems that include one or more compounds) include binary and ternary photoinitiators. In one example, a ternary photoinitiator may include an iodonium salt, a photosensitizer, and an electron donor compound as described in U.S. Pat. No. 5,545,676 (Palazzotto et al.). Examples of iodonium salts include diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Examples of photosensitizers include monoketones and diketones that absorb some light within a range of about 400 nanometers to 520 nanometers, preferably 450 to 500 nanometers. Preferred are alpha diketones that absorb light within these ranges. Examples of such photosensitizers include camphoroquinone, benzil, furil, 3,3,6,6-tetramethylcyclohexanedione, phenanthraquinone, 1-phenyl-1,2-propanedione, and other 1-aryl-1-alkyl-1,2-ethanediones, and cyclic alpha diketones. Most preferred is camphoroquinone. Preferred electron donor compounds include substituted amines, e.g., ethyl dimethylaminobenzoate.

The photoinitiator, when utilized, should be present in an amount sufficient to provide the desired rate of polymerization. This amount will be dependent in part on the light source, the thickness of the layer to be exposed to radiant energy, and the extinction coefficient of the photoinitiator.

In some embodiments, each part of the multi-part hardenable dental composition described herein may have a viscosity which is balanced with respect to the other parts of the composition. In some embodiments, including any one of the above embodiments, the first part and second part may each, independently, have a viscosity not less than 6 pascal-second (Pa·s) and not greater than 100 Pa·s. In some embodiments, the ratio of second part to first part viscosity is 1:0.06 to 1:13. 1:0.6 to 1:3.5, or 1:0.9 to 1:1.6.

For purposes of the present disclosure, viscosity is as measured on a TA instrument (AR G2), at room temperature, at a shear rate of 20/s, with a simple shear method.

In some embodiments, upon mixing of the first part and the second part, the hardenable dental composition may have a set time of less than 10 minutes, less than 7 minutes, or less than 5 minutes. As used herein, “set time” refers to the time at which sufficient curing has occurred, at room temperature and without heat or light activation, so that essentially the composition's final cured-state properties are obtained.

The multi-part hardenable compositions of the present disclosure may be formed by combining the first part and the second part. Any conventional mixing or combination techniques may be employed. For example, the first and second parts can be dispensed onto a mix pad and mixed by hand, can be mixed using an automix tip, or can be mixed by a rotary instrument

In some embodiments, the methods, devices, and compositions described herein are well suited for a number of dental applications, such as, for example, a luting cement used to anchor or hold a prosthetic device (e.g., crown, bridge, inlay, onlay, post, abutment, veneer, prosthetic tooth, and the like) in place in the mouth; a restorative or filler material used, for example, for filling a cavity; a thin film used, for example, as a liner on dentin and enamel or a sealant or sealing material on enamel; an orthodontic bracket adhesive; a band cement; and the like. In some embodiments, the multi-part hardenable dental composition is selected from the group consisting of a liner material, a luting material, a restorative material, an endodontic material, and a sealing material. For certain embodiments, including any one of the above embodiments, the multi-part hardenable dental composition is an orthodontic bracket adhesive material or band cement.

In some embodiments, the present disclosure provides methods of making and using the multi-part hardenable compositions described herein. For example, the hardenable compositions of the present disclosure (after mixing) can be used in methods of adhering or cementing (either intraorally or extraorally) a dental article (e.g., crown, bridge, orthodontic appliance) to a tooth or bone, as well as in methods of filling a tooth.

Objects and advantages of this disclosure are further illustrated by the following examples, but the particular materials and amounts thereof recited in these examples, as well as other conditions and details, should not be construed to unduly limit this disclosure.

### EXAMPLES

TABLE 1
Materials List

| ABBREVIATION/ | | |
|---|---|---|
| NAME | DESCRIPTION | SOURCE |
| BHT | butylated hydroxytoluene (>99%, FCC, FG); | Sigma-Aldrich |
| | CAS# 128-37-0 | |
| Calcium silicate | Calcium silicate; CAS# 1344-95-2 (12-22% | Sigma-Aldrich |
| | Ca (as CaO) basis, >97% SiO2 basis) | |
| tricalcium | tricalcium phosphate, Ca₃(PO₄)₂; | Sigma-Aldrich |
| phosphate | CAS# 7758-87-4 | |
| potassium | potassium persulfate, milled (K₂S₂O₈); | Sigma-Aldrich |
| persulfate | CAS 7727-21-1 | |
| CPQ | Camphorquinone: CAS# 10373-78-1 | Sigma-Aldrich |
| DMAPE | 2-[4-(Dimethylamino)phenyl]ethanol, | Sigma-Aldrich |
| | CAS# 50438-75-0 | |
| EDMAB | Ethyl 4-(N,N-dimethylamino)benzoate; | Sigma-Aldrich |
| | CAS# 10287-53-3 | |
| 85% H3PO4 | 85% (w/w) phosphoric acid in water solution; | Sigma-Aldrich |
| | CAS# 7664-38-2 | |
| 36% HCl | 36% (w/w) hydrochloric acid in water | MilliporeSigma |
| solution | solution; (CAS# 7647-01-0 | |
| 60% HNO3 | 60% (w/w) nitric acid in water solution; | EMD Millipore |
| solution | CAS# 7697-37-2 | Corporation |
| 50% H2SO4 | 50% (w/w) sulfuric acid in water solution; | J. T. Baker |
| solution | CAS# 7664-93-9 | |
| GDMA | glycerol dimethacrylate | Sigma-Aldrich |
| HEMA | 2-hydroxyethyl methacrylate | Sigma-Aldrich |
| MEHQ | 4-methoxyphenol, CAS# 150-76-5 | Sigma-Aldrich |
| HEMA with | HEMA doped with 150 ppm MEHQ inhibitor | 3M Prepared internally |
| MEHQ | | |
| fumed silica | AEROSIL R812S, hydrophobic fumed silica | Evonik Industries |
| Zr—si filler | Silane treated zirconia-silica filler | Prepared as described in |
| | | U.S. Pat. No. 6,818,682, |
| | | col. 11, line 41 through |
| | | col. 12, line 10. |
| S/T coarse FAS | Silane treated fluoroaluminosilicate (FAS) | Silane treated as described |
| filler | reactive glass filler; having a particle | in U.S. Pat. No. |
| | size of about 3 micrometers. | 7,173,074, column 13 |
| | | (Mo-Sci Corp., Columbia, |
| | | MO) |
| S/T fine FAS | Silane treated fluoroaluminosilicate (FAS) | Silane treated as described |
| filler | reactive glass filler; having a particle | in U.S. Pat. No. |
| | size of about 1 micrometers. | 7,173,074, column 13 |
| | | (Mo-Sci Corp., Columbia, |
| | | MO) |
| ALD coated | Portland Cement Cores Encapsulated Using | 3M prepared internally; |
| Portland cement | Atomic Layer Deposition (ALD) | prepared according to |
| | | Example 23 of U.S. patent |
| | | application No. |
| | | US2019/0388355A1 |
| TiO₂ | Titanium oxide; CAS# 13463-67-7 | DUPONT |
| YbF3 filler | Yitterbium fluoride 100 nm radiopacity | Sukgyung AT Co. Ltd - |
| | filler | Korea |
| AA:ITA | Copolymer made from a 4:1 mole ratio of | 3M prepared internally; |
| | acrylic acid:itaconic acid, | prepared according to |
| | | Example 3 of U.S. Pat. |
| | | No. 5,130,347 (Mitra), |
| | | MW (average) = 106,000; |
| | | polydispersity ρ = 4.64. |
| VBCP | An acrylic-itaconic acid copolymer with | 3M prepared internally; |
| | pendant methacrylate side group made by | prepared according to the |
| | reacting AA:ITA copolymer with sufficient | dry polymer preparation |
| | IEM to convert 16 mole percent of the | of Example 11 of U.S. |
| | acid groups of the copolymer to pendent | Pat. No. 5,130,347. |
| | methacrylate groups. | |

### “Paste B” Preparations for Resin-Modified Glass Ionomer (RMGI) Dental Compositions

Resin-Modified Glass Ionomer (RMGI) cements are two-part systems that when the two parts are combined, they react and harden (cure). The RMGI examples below were prepared as paste-paste type of RMGI, (Paste “A” and Paste “B”) two-part reactive systems.

Comparative Examples CEx.B1 and CEx.B32 lacked a strong acid and so formed a gel after sifting overnight, even though upon initial mixing they were in solution. Therefore, CEx.B1 and CEx.B32 were not acceptable to be used as Paste “B” in an RMGI composition.

TABLE 2
Paste “B” portion of RMGI Cement Two-Part System

| | RMGI Paste “B” | | | |
|---|---|---|---|---|
| | CEx. B1 | CEx. B2 | EXB1 | EXB2 |
| | Paste B-C1 | Paste B-C2 | Paste B1 | Paste B2 |
| Components | grams | grams | grams | Grams |
| DI water | 20 | 27.5 | 27.5 | 27.45 |
| EDMAB | 1.2 | — | — | — |
| DMAPE | — | 2.5 | 2.5 | 1.0 |
| MEHQ | — | 0.01 | 0.01 | — |
| 85% H3PO4 | — | — | 1.75 | 0.8 |
| VBCP | 28 | 28 | 28 | 30 |
| HEMA with MEHQ | 15 | 9.75 | 8 | 8 |
| Formed Gel? * | Yes | Yes | No | No |

“Yes” = formed a gel overnight; NOT acceptable as a paste “B” for RMGI.
“No” = did not form a gel overnight, acceptable as a paste “B” for RMGI.

TABLE 3
Paste “B” Portion with Various Acids for Use in RMGI

| | RMGI Paste “B” | | | | |
|---|---|---|---|---|---|
| | EXB3 | EXB4 | EXB5 | EXB6 | EXB7 |
| | Paste B3 | Paste B4 | Paste B5 | Paste B6 | Paste B7 |
| Components | grams | grams | grams | grams | grams |
| DI water | 33 | 19.4 | 17 | 17 | 17 |
| DMAPE | 2.5 | 1.0 | 1.5 | 1.5 | 1.5 |
| MEHQ | 0.01 | — | — | — | — |
| 85% H3PO4 | 1.75 | 0.8 | — | — | — |
| 36% HCl | — | — | 0.95 | — | — |
| solution | | | | | |
| 60% HNO3 | — | — | — | 1.0 | —- |
| solution | | | | | |
| 50% H2SO4 | — | — | — | — | 1.85 |
| solution | | | | | |
| BHT | — | — | 0.05 | 0.05 | 0.05 |
| VBCP | 30 | 28 | 28 | 28 | 28 |
| HEMA | — | 15 | 17 | 17 | 17 |
| with MEHQ | | | | | |
| Formed Gel? | No | No | No | No | No |

“No” = did not form a gel overnight, acceptable as a paste “B” for RMGI.

### “Paste A” Preparations for Resin-Modified Glass Ionomer (RMGI) Dental Compositions

TABLE 4
Paste “A” Portion for a RMGI Cement Two-Part System

| | RMGI Paste “A” | | | | | | |
|---|---|---|---|---|---|---|---|
| | EXA1 | EXA2 | EXA3 | EXA4 | EXA5 | EXA6 | EXA7 |
| | Paste | Paste | Paste | Paste | Paste | Paste | Paste |
| | A1 | A2 | A3 | A4 | A5 | A6 | A7 |
| Components | % | % | % | % | % | % | % |
| HEMA | — | 14 | 12 | 12 | 12 | 14 | 20.3 |
| GDMA | 32 | 14 | 12 | 12 | 12 | 14 | 8.1 |
| BHT | — | — | — | 0.05 | 0.1 | — | 0.03 |
| potassium persulfate | 2 | 2 | 2 | 2 | 2 | 2 | 1.5 |
| tricalcium phosphate | — | — | 0.85 | — | 0.75 | 0.85 | — |
| ALD coated Portland | — | — | — | 7 | — | — | — |
| cement | | | | | | | |
| fumed silica | 4 | 6 | 3 | 3 | 3 | 5 | 2 |
| S/T coarse FAS filler | 52 | 64 | 70 | 63.8 | 70 | 64 | 30 |
| S/T fine FAS filler | — | — | — | — | — | — | 38 |
| Calcium silicate | 10 | — | — | — | — | — | — |
| CPQ | — | — | 0.15 | 0.15 | 0.15 | 0.15 | 0.10 |
| Total | 100 | 100 | 100 | 100 | 100 | 100 | 100 |

TABLE 5
Additional Paste “B” Compositions for RMGI Cement Two-Part System

| | RMGI Paste “B” | | | | | |
|---|---|---|---|---|---|---|
| | EXB8 | EXB9 | EXB10 | EXB11 | EXB12 | EXB13 |
| | Paste B8 | Paste B9 | Paste B10 | Paste B11 | Paste B12 | Paste B13 |
| Components | % | % | % | % | % | % |
| DI water | 33.5 | 27.5 | 33.0 | 27.45 | 27.0 | 27.0 |
| DMAPE | 2.5 | 2.5 | 2.5 | 1.0 | 0.5 | 0.5 |
| MEHQ | 0.01 | 0.01 | 0.01 | — | — | — |
| 85% H3PO4 | 1.75 | 1.75 | 1.75 | 0.8 | 0.8 | 0.4 |
| TiO2 | — | — | 0.5 | 0.5 | 0.4 | 0.8 |
| Zr—si filler | 32 | 32 | 32 | 32 | 35 | 35 |
| fumed silica | 0.24 | 0.24 | 0.24 | 0.25 | 0.3 | 0.3 |
| VBCP | 30 | 28 | 30 | 30 | 28 | 28 |
| HEMA with MEHQ | — | 8 | — | 8 | 8 | 8 |
| Total | 100 | 100 | 100 | 100 | 100 | 100 |

TABLE 6
Additional Paste “B” Compositions
for RMGI Cement Two-Part System

| | RMGI Paste “B” | | | |
|---|---|---|---|---|
| | EXB14 | EXB15 | EXB16 | EXB17 |
| | Paste B14 | Paste B15 | Paste B16 | Paste B17 |
| Components | % | % | % | % |
| DI water | 27.0 | 19.4 | 17.0 | 17.0 |
| DMAPE | 0.5 | 1.0 | 1.0 | 1.0 |
| 85% H3PO4 | 0.6 | 0.8 | 0.8 | 0.8 |
| TiO2 | 0.6 | 0.5 | 0.5 | 0.4 |
| Zr—si filler | 13 | 35 | 35 | 35 |
| fumed silica | 0.3 | 0.3 | 0.65 | 0.73 |
| BHT | — | — | 0.05 | 0.07 |
| YbF3 filler | 22 | — | — | — |
| VBCP | 28 | 28 | 28 | 28 |
| HEMA | | | | |
| with MEHQ | 8 | 15 | 17 | 17 |
| Total | 100 | 100 | 100 | 100 |

TABLE 7
Additional Paste “B” Compositions
for RMGI Cement Two-Part System

RMGI Paste “B”

| | EXB18 | EXB19 | EXB20 |
|---|---|---|---|
| | Paste B18 | Paste B19 | Paste B20 |
| Components | % | % | % |
| DI water | 17.0 | 17 | 17 |
| DMAPE | 1.5 | 1.5 | 1.5 |
| 36% HCl solution | 0.95 | — | — |
| 60% HNO3 solution | — | 1.0 | — |
| 50% H2SO4 solution | — | — | 1.85 |
| Zr—si filler | 35 | 34.95 | 34.1 |
| fumed silica | 0.5 | 0.5 | 0.5 |
| BHT | 0.05 | 0.05 | 0.05 |
| VBCP | 28 | 28 | 28 |
| HEMA with MEHQ | 17 | 17 | 17 |
| Total | 100 | 100 | 100 |

### Preparation of Resin-Modified Glass Ionomer (RMGI) Cement Examples

Two-part Resin-Modified Glass Ionomer (RMGI) cements were prepared using the above Example Pastes “A” and Pastes “B.” The selected Paste “A” and Paste “B” samples were mixed by hand for 20 seconds in a 6:5 part ratio (A:B respectively), on a mixing pad using a dental spatula. The mixed examples were then placed in a 37° C. oven to cure and harden (set). The mixtures were tapped with the spatula to verify they were set, the set time was recorded and is reported in Table 8, below.

TABLE 8
Resin-Modified Glass Ionomer (RMGI) Cement
Examples prepared with Paste A and Paste B

| | | | Set time |
|---|---|---|---|
| RMGI | | | (minute′ |
| Example | Paste A | Paste B | second″) |
| EX-RMGI-1 | EXA2 Paste A2 | EXB8 Paste B8 | 3′00″ |
| EX-RMGI-2 | EXA2 Paste A2 | EXB9 Paste B9 | 3′00″ |
| EX-RMGI-3 | EXA1 Paste A1 | EXB9 Paste B9 | 7′00″ |
| EX-RMGI-4 | EXA2 Paste A2 | EXB10 Paste B10 | 3′00″ |
| EX-RMGI-5 | EXA6 Paste A6 | EXB10 Paste B10 | 3′00″ |
| EX-RMGI-6 | EXA6 Paste A6 | EXB11 Paste B11 | 3′00″ |
| EX-RMGI-7 | EXA6 Paste A6 | EXB12 Paste B12 | 3′30″ |
| EX-RMGI-8 | EXA6 Paste A6 | EXB14 Paste B14 | 3′20″ |
| EX-RMGI-9 | EXA6 Paste A6 | EXB13 Paste B13 | 3′20″ |
| EX-RMGI-10 | EXA3 Paste A3 | EXB11 Paste B11 | 3′00″ |
| EX-RMGI-11 | EXA3 Paste A3 | EXB15 Paste B15 | 3′05″ |
| EX-RMGI-12 | EXA4 Paste A4 | EXB17 Paste B17 | 3′10″ |
| EX-RMGI-13 | EXA5 Paste A5 | EXB15 Paste B15 | 3′20″ |
| EX-RMGI-14 | EXA3 Paste A3 | EXB16 Paste B16 | 5′30″ |
| EX-RMGI-15 | EXA5 Paste A5 | EXB11 Paste B11 | 3′30″ |
| EX-RMGI-16 | EXA5 Paste A5 | EXB9 Paste B9 | 2′50″ |
| EX-RMGI-17 | EXA4 Paste A4 | EXB9 Paste B9 | 2′50″ |
| EX-RMGI-18 | EXA4 Paste A4 | EXB11 Paste B11 | 3′00″ |
| EX-RMGI-19 | EXA4 Paste A4 | EXB8 Paste B8 | 2′50″ |
| EX-RMGI-20 | EXA5 Paste A5 | EXB18 Paste B18 | 3′50″ |
| EX-RMGI-21 | EXA5 Paste A5 | EXB19 Paste B19 | 3′55″ |
| EX-RMGI-22 | EXA5 Paste A5 | EXB20 Paste B20 | 4′00″ |

Stability of Resin-Modified Glass Ionomer (RMGI) Cements Prepared with Paste A and Paste B Components Stored Over Time at Elevated Temperature.

A first stability experiment was conducted to test the stability of select Example Pastes B which had been stored 45° C. for 26 days in polypropylene syringe clicker. Commercially available 3M product RelyX™ Luting Plus Cement Clicker™ dispensers were emptied and used to provide the storage containers for select Example Pastes B. One cylinder of the clicker was filled the experimental paste B and stored at 45° C. for 26 days. After the storage period, the Example Pastes B were extruded through the syringe and dispenser tip to verify Paste B was still extrudable, and visually evaluated for any indication of premature self-cure; results are reported in Table 9.

Additionally, each of the Example Pastes B which had been stored at 450 for 26 days were then mixed with Example Paste EXA6 Paste A6 to form a Resin-Modified Glass Ionomer (RMGI). Paste A and Paste B were mixed by hand for 20 seconds in a 6:5 part ratio (A:B, respectively; 0.42 gram of Paste A mixed with 0.35 gram Paste B), on a mixing pad using a dental spatula. The mixed examples were then placed in a 37° C. oven to cure and harden (set). The mixtures were tapped with the spatula to verify they reacted and set. The set time for each RMGI Example was recorded and is reported in Table 9, below.

TABLE 9
Resin-Modified Glass Ionomer (RMGI) Cement Examples prepared
with Example Pastes B stored at 45° C. for 26 days

| | | | | Set time |
|---|---|---|---|---|
| | Paste B | Paste B | | with EXA6 |
| | stored @ | Extrudable | Paste B | Paste A6 |
| RMGI | 45° C. for | from | Premature | (minute′ |
| Example | 26 days | syringe? | Self-cure? | second″) |
| EX-RMGI-23 | EXB10 Paste B10 | Yes | No | 2′40″ |
| EX-RMGI-24 | EXB11 Paste B11 | Yes | No | 2′40″ |
| EX-RMGI-25 | EXB12 Paste B12 | Yes | No | 2′45″ |
| EX-RMGI-26 | EXB13 Paste B13 | Yes | No | 3′05″ |
| EX-RMGI-27 | EXB14 Paste B14 | Yes | No | 3′10″ |

A second stability experiment was conducted to test the stability of select Example Pastes B stored at 45° C. for 2 months in polypropylene syringe clicker. Commercially available 3M product RelyX™ Luting Plus Cement Clicker™ dispensers were emptied and used to provide the storage containers for select Example Pastes B. One cylinder of the clicker was filled the experimental paste B and stored at 45° C. for 2 months. After the storage period, the Example Pastes B were extruded through the syringe and dispenser tip to verify Paste B was still extrudable, and visually evaluated for any indication of premature self-cure; results are reported in Table 10.

Additionally, each Example Pastes B that had been stored at 45° C. for 2 months were then mixed with Example EXA7 Paste A7, which had been stored for 32 months at room temperature (ambient lab conditions), to form a Resin-Modified Glass Ionomer (RMGI). This aged Example EXA7 Paste A7 and each Paste B were mixed by hand for 20 seconds in a 6:5 part ratio (A:B, respectively; 0.42 gram of Paste A mixed with 0.35 gram Paste B), on a mixing pad using a dental spatula. The mixed examples were then placed in a 37° C. oven to cure and harden (set). The mixtures were tapped with the spatula to verify they reacted and set; the set time for each example was recorded and is reported in Table 10.

TABLE 10
Resin-Modified Glass Ionomer (RMGI) Cement Examples prepared
with Example Pastes B stored at 45° C. for 2 months
and Paste A stored for 32 months at Room Temperature

| | | | | Set time |
|---|---|---|---|---|
| | Paste B | Paste B | | with EXA7 |
| | stored @, | Extrudable | Pate B | Paste A7* |
| RMGI | 45° C. for | from | Premature | (minute′ |
| Example | 2 months | syringe? | Self-cure? | second″) |
| EX-RMGI-28 | EXB10 Paste B10 | Yes | No | 3′10″ |
| EX-RMGI-29 | EXB11 Paste B11 | Yes | No | 3′15″ |
| EX-RMGI-30 | EXB12 Paste B12 | Yes | No | 3′30″ |
| EX-RMGI-31 | EXB13 Paste B13 | Yes | No | 3′05″ |
| EX-RMGI-32 | EXB14 Paste B14 | Yes | No | 3′25″ |

*EXA7 Paste A7 stored for 32 months at RT

A third stability experiment was conducted in a manner like RMGI Examples EX-RMGI-28-32, described above, except using EXA5 Paste A5, which had been stored at 45° C. for 1 month and EXB13 Paste B13, which had been stored at 45° C. for 2 months. The results are reported in Table 11.

TABLE 11
Resin-Modified Glass Ionomer (RMGI) Cement Examples
prepared with Example Paste B stored at 45° C.
for 2 months and Paste A stored at 45° C. for 1 month

| | | | | Set time |
|---|---|---|---|---|
| | Paste B | Paste B | | with EXA5 |
| | stored @ | Extrudable | Pate B | Paste A5* |
| RMGI | 45° C. for | from | Premature | (minute′ |
| Example | 2 months | syringe? | Self-cure? | second″) |
| EX-RMGI-33 | EXB13 Paste B13 | Yes | No | 3′20″ |

*EXA5 Paste A5 stored at 45° C. for 1 month

1. A multi-part hardenable dental composition comprising:
a first part comprising:
a mono- and/or multi-functional component having one or more ethylenically unsaturated group(s),
acid-reactive glass particles; and
an oxidizing agent; and
a second part comprising:
a mono- and/or multi-functional component having one or more ethylenically unsaturated group(s),
a water miscible polyacid;
a reducing agent comprising an aromatic tertiary amine;
an acid having a pKa less than that of carboxylic acid; and
non-reactive filler.

2. The multi-part hardenable dental composition of claim 1, wherein the oxidizing agent comprises a peroxide.

3. The multi-part hardenable dental composition of claim 1, wherein the oxidizing agent comprises a persulfate.

4. The multi-part hardenable dental composition of claim 1, wherein the oxidizing agent comprises potassium persulfate.

5. The multi-part hardenable dental composition of claim 1, wherein the oxidizing agent is present in the first part in an amount of between 0.1 and 4.0 wt. %, based on the total weight the first part.

6. The multi-part hardenable dental composition of claim 1, wherein the acid-reactive glass comprises fluoroaluminosilicate (FAS) glass.

7. The multi-part hardenable dental composition of claim 1, wherein the acid-reactive glass is present in the first part in an amount of between 3 and 60 wt. %, based on the total weight the first part.

8. The multi-part hardenable dental composition of claim 1, wherein the polyacid is of the formula: 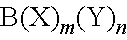
wherein B is a hydrocarbon backbone, X is —COOH, Y is an ethylenically unsaturated group, m is at least 2, n is at least 1, and Y is attached to B via an amide linkage.

9. The multi-part hardenable dental composition of claim 1, wherein the polyacid comprises the reaction product of (i) a polymer selected from the group consisting of polyacrylic acids, copolymers of acrylic and itaconic acids, copolymers of acrylic and maleic acids, copolymers of methyl vinyl ether and maleic anhydride or maleic acid, copolymers of ethylene and maleic anhydride or maleic acid, copolymers of styrene and maleic anhydride or maleic acid, and a combination thereof, and (ii) a coupling compound selected from the group consisting of acryloyl chloride, methacryloyl chloride, vinyl azalactone, allyl isocyanate, 2-hydroxyethyl methacrylate, 2-aminoethylmethacrylate, and 2-isocyanatoethyl methacrylate.

10. The multi-part hardenable dental composition of claim 1, wherein the polyacid is present in the second part in an amount of between 5 and 35 wt. %, based on the total weight the second part.

11. The multi-part hardenable dental composition of claim 1, wherein the aromatic tertiary amine has a structural formula as follows: 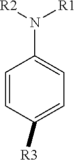
where R1, R2, and R3 are, independently, (i) a hydrogen atom or (ii) an alkyl group, alkyl alcohol group, an alkyl group that includes an ester or amide linkage, an ester group, an amide group, or a urethane group, having from 1-8 carbon atoms.

12. The multi-part hardenable dental composition of claim 1, wherein the aromatic tertiary amine is present in the second part in an amount of between 0.25 and 5.0 wt. %, based on the total weight the second part.

13. The multi-part hardenable dental composition of claim 1, wherein the acid comprises phosphoric acid, sulfate acid, nitric acid, hydrochloride acid, or one or more monomers containing one or more phosphate groups.

14. The multi-part hardenable dental composition of claim 1, wherein water is present in the second part in an amount of between 2 and 30 wt. %, based on the total weight the second part.

15. The multi-part hardenable dental composition of claim 1, wherein the first part, the second part, or both of the first and second parts comprise nonreactive filler in an amount of 1 to 40 weight percent, based upon the total weight of the part which includes the nonreactive filler, and wherein the nonreactive filler comprises inorganic material, crosslinked organic material, and a combination thereof.

16. The multi-part hardenable dental composition of claim 1, wherein the second part includes the non-reactive filler in an amount of 30 to 40 weight percent, based upon the total weight of the second part.

17. The multi-part hardenable dental composition of claim 1, wherein the mono- and/or multi-functional components of the first and second part are liquid at room temperature and water miscible.

18. The multi-part hardenable dental composition of claim 1, wherein the first part comprises both mono- and multi-functional components having ethylenically unsaturated group(s).

19. The multi-part hardenable dental composition of claim 1, wherein the mono- and multi-functional components having ethylenically unsaturated group(s) of the first part and second part, independently, are present in their respective parts in an amount of between 3 and 40 wt. %, based on the total weight the respective part.

20. (canceled)

21. The multi-part hardenable dental composition of claim 1, wherein the mono- and multi-functional components having ethylenically unsaturated group(s) of the first part or the second part comprise 2-hydroxyethyl methacrylate, glycerol mono(meth)acrylate, or a sugar methacrylate.

22-25. (canceled)
